(19) United States
(12) Patent Application Publication
(10) Pub. No.: US 2007/0276234 A1
(43) Pub. Date: Nov. 29, 2007

(54) Systems and Methods for Intraoperative Targeting

(75) Inventor: Ramin Shahidi, Palo Alto, CA (US)

Correspondence Address: Mani Adeli;Slattler Johansen & Adeli LLP, 1875 Century Park East Suite 1360, Los Angeles, CA, 90067, US

(73) Assignee: The Board of Trustees of the Leland Stanford Junior University, Palo Alto, CA (US)

(21) Appl. No.: 10/576,632

(22) PCT Filed: Oct. 21, 2004
(86) PCT No.: PCT/US04/35014

§ 371 Date: Apr. 26, 2007

Related U.S. Application Data
(60) Provisional application No. 60/513,157, filed on Oct. 21, 2003.

(30) Foreign Application Priority Data
Jan. 26, 2004 (US) 10764650
Jan. 26, 2004 (US) 10764651

(51) Int. Cl. A61B 8/00

(52) U.S. Cl. 600/437

(57) ABSTRACT

The method of some embodiments of the invention assists a user in guiding a medical instrument to a subsurface target site in a patient. This method generates at least one intraoperative ultrasonic images. The method indicates a target site on the ultrasonic image(s). The method determines 3-D coordinates of the target site in a reference coordinate system. The method (1) tracks the position of the instrument in the reference coordinate system, (2) projects onto a display device a view field as seen from the position with respect to the tool in the reference coordinate system, and (3) projects onto the displayed view field indicia of the target site corresponding to the position. In some embodiments, the field of view is a view not only from the position of the instrument but also from a known orientation of the instrument in the reference coordinate system. By observing the indicia, the user can guide the instrument toward the target site by moving the instrument so that the indicia are placed or held in a given state in the displayed field of view.

### BACKGROUND

In recent years, the medical community has been increasingly focused on minimizing the invasiveness of surgical procedures. Advances in imaging technology and instrumentation have enabled procedures using minimally-invasive surgery with very small incisions. Growth in this category is being driven by a reduction in morbidity relative to traditional open procedures, because the smaller incisions minimize damage to healthy tissue, reduce patient pain, and speed patient recovery. The introduction of miniature CCD cameras and their associated micro-electronics has broadened the application of endoscopy from an occasional biopsy to full minimally-invasive surgical ablation and aspiration.

Minimally-invasive endoscopic surgery offers advantages of a reduced likelihood of intraoperative and post-operative complications, less pain, and faster patient recovery. However, the small field of view, the lack of orientation cues, and the presence of blood and obscuring tissues combine to make video endoscopic procedures in general disorienting and challenging to perform. Modern volumetric surgical navigation techniques have promised better exposure and orientation for minimally-invasive procedures, but the effective use of current surgical navigation techniques for soft tissue endoscopy is still hampered by two difficulties: (1) accurately tracking all six degrees of freedom (DOF) on a flexible endoscope within the body, and (2) compensating for tissue deformations and target movements during an interventional procedure.

To illustrate, when using an endoscope, the surgeon's vision is limited to the camera's narrow field of view and the lens is often obstructed by blood or fog, resulting in the surgeon suffering a loss of orientation. Moreover, endoscopes can display only visible surfaces and it is therefore often difficult to visualize tumors, vessels, and other anatomical structures that lie beneath opaque tissue (e.g., targeting of pancreatic adenocarcinomas via gastro-intestinal endoscopy, or targeting of submucosal lesions to sample peri-intestinal structures such as masses in the liver, or targeting of subluminal lesion in the bronchi).

Recently, image-guided therapy (IGT) systems have been introduced. These systems complement conventional endoscopy and have been used predominantly in neurological, sinus, and spinal surgery, where bony or marker-based registration can provide adequate target accuracy using pre-operative images (typically 1-3 mm). While IGT enhances the surgeon's ability to direct instruments and target specific anatomical structures, in soft tissue these systems lack sufficient targeting accuracy due to intra-operative tissue movement and deformation. In addition, since an endoscope provides a video representation of a 3D environment, it is difficult to correlate the conventional, purely 2D IGT images with the endoscope video. Correlation of information obtained from intra-operative 3D ultrasonic imaging with video endoscopy can significantly improve the accuracy of localization and targeting in minimally-invasive IGT procedures.

Until the mid 1990's, the most common use of image guidance was for stereotactic biopsies, in which a surgical trajectory device and a frame of reference were used. Traditional frame-based methods of stereotaxis defined the intracranial anatomy with reference to a set of fiducial markers, which were attached to a frame that was screwed into the patient's skull. These fiducials were measured on pre-operative tomographic (MRI or CT) images.

A trajectory-enforcement device was placed on top of the frame of reference and used to guide the biopsy tool to the target lesion, based on prior calculations obtained from pre-operative data. The use of a mechanical frame allowed for high localization accuracy, but caused patient discomfort, limited surgical flexibility, and did not allow the surgeon to visualize the approach of the biopsy tool to the lesion.

There has been a gradual emergence of image guided techniques that eliminate the need for the frame altogether. The first frameless stereotactic system used an articulated robotic arm to register pre-operative imaging with the patient's anatomy in the operating room. This was followed by the use of acoustic devices for tracking instruments in the operating environment. The acoustic devices eventually were superceded by optical tracking systems, which use a camera and infrared diodes (or reflectors) attached to a moving object to accurately track its position and orientation. These systems use markers placed externally on the patient to register pre-operative imaging with the patient's anatomy in the operating room. Such intra-operative navigation techniques use pre-operative CT or MR images to provide localized information during surgery. In addition, all systems enhance intra-operative localization by providing feedback regarding the location of the surgical instruments with respect to 2D preoperative data.

Until recently, volumetric surgical navigation has been limited by the lack of the computational power required to produce real-time 3D images. The use of various volumetric imaging modalities has progressed to permit the physician to visualize and quantify the extent of disease in 3D in order to plan and execute treatment. Systems are currently able to provide real-time fusion of pre-operative 3D data with intraoperative 2D data images from video cameras, ultrasound probes, surgical microscopes, and endoscopes. These systems have been used predominantly in neurological, sinus, and spinal surgery, where direct access to the pre-operative data plays a major role in the execution of the surgical task. This is despite the fact that, because of movement and deformation of the tissue during the surgery, these IGT procedures tend to lose their spatial registration with respect to the pre-operatively acquired image.

### SUMMARY

The method of some embodiments of the invention assists a user in guiding a medical instrument to a subsurface target site in a patient. This method generates at least one intraoperative ultrasonic images. The method indicates a target site on the ultrasonic image(s). The method determines 3-D coordinates of the target site in a reference coordinate system. The method (1) tracks the position of the instrument in the reference coordinate system, (2) projects onto a display device a view field as seen from the position with respect to the tool in the reference coordinate system, and (3) projects onto the displayed view field indicia of the target site corresponding to the position. In some embodiments, the field of view is a view not only from the position of the instrument but also from a known orientation of the instrument in the reference coordinate system. By observing the indicia, the user can guide the instrument toward the target site by moving the instrument so that the indicia are placed or held in a given state in the displayed field of view.

### BRIEF DESCRIPTION OF THE DRAWINGS

The novel features of the invention are set forth in the appended claims. However, for purpose of explanation, several embodiments of the invention are set forth in the following figures.

FIGS. 1-2 show exemplary flowcharts of the operation of the system of some embodiments of the invention.

FIGS. 3-4 shows exemplary user interface displays of the system of some embodiments of the invention.

FIGS. 5-6 shows exemplary operating set-up arrangements in accordance with one aspect of the system.

### DETAILED DESCRIPTION OF THE INVENTION

In the following description, numerous details are set forth for purpose of explanation. However, one of ordinary skill in the art will realize that the invention may be practiced without the use of these specific details. In other instances, well-known structures and devices are shown in block diagram form in order not to obscure the description of the invention with unnecessary detail.

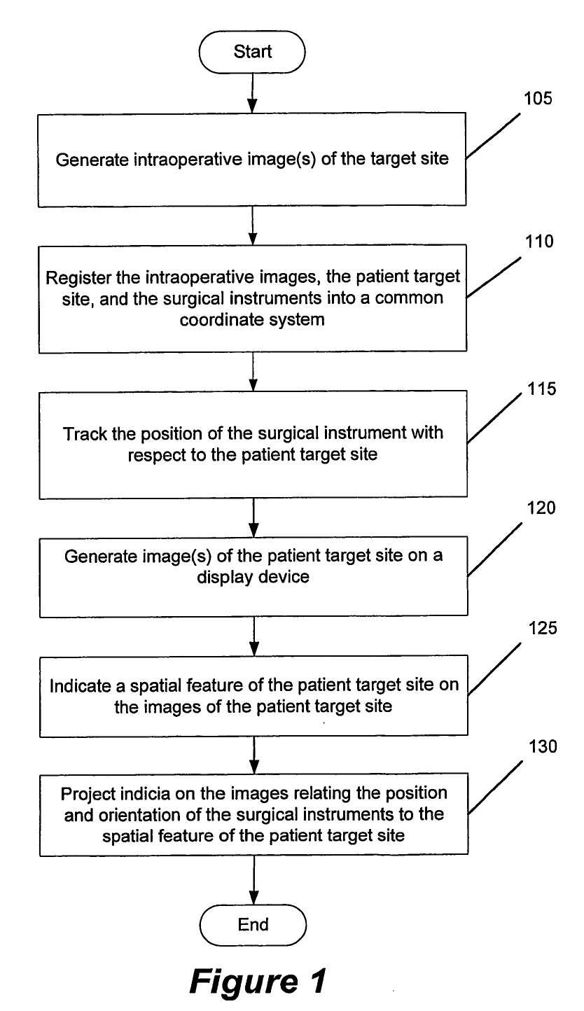

FIG. 1 illustrates a process 100 of some embodiments of the invention. This process guides a medical instrument to a desired position in a patient. As shown in this figure, the process 100 initially acquires (at 105) one or more intraoperative images of the target site. Next, the process 100 registers (at 110) the intraoperative images, the patient target site, and the surgical instruments into a common coordinate system.

The patient, the imaging source(s) responsible for the intraoperative images and surgical tool must all be placed in the same frame of reference (in registration). This can be done by a variety of methods, three of which are described below. First, a wall-mounted tracking device can be used to track the patient, imaging source(s), and the surgical tool (e.g., endoscope). Second, only the position of the tool can be tracked. Under such an approach, the tool can be placed in registration with the patient and imaging source by touching the tool point to fiducials on the body and to the positions of the imaging source(s). Thereafter, if the patient moves, the device could be registered by tool-to-patient contacts. That is, once the images are made, from known coordinates, it is no longer necessary to further track the position of the image source(s).

Third the patient and image sources are placed in registration by fiducials on the patient and in the images, or alternatively, by placing the imaging device at known coordinates with respect to the patient. The patient and tool are placed in registration by detecting the positions of fiducials with respect to the tool, e.g., by using a detector on the tool for detecting the positions of the patient fiducials. Alternatively, the patient and an endoscope tool can be placed in registration by imaging the fiducials in the endoscope, and matching the imaged positions with the position of the endoscope.

After the registration operation at 110, the process 100 tracks (at 115) the position of the surgical instrument with respect to the patient target site. In some embodiments, a magnetic tracking system is used to track the endoscope for navigation integration in one implementation. The system provides a magnetic transducer into the working channel at the endoscope tip, positioning the field generator so that the optimal sensing volume encompasses the range of sensor positions. In one implementation that provides for six degrees of freedom (6 DOF), a miniaturized magnetic tracking system with metal insensitivity can be used. The tracking system may be calibrated using a calibration jig. A calibration target is modified from a uniform to a non-uniform grid of points by reverse-mapping the perspective transform, so that the calibration target point density is approximately equal throughout the endoscope image. The calibration jig is waterproofed and designed to operate in a submerged environment. Where appropriate, calibration will be performed while the jig is immersed in a liquid with refractive properties similar to the operating environment.

In one embodiment, an ultrasound calibration system can be used for accurate reconstruction of volumetric ultrasound data. An optical tracking system is used to measure the position and orientation of a tracking device that will be attached to the ultrasound probe. A spatial calibration of intrinsic and extrinsic parameters of the ultrasound probe is performed. These parameters are used to transform the ultrasound image into the co-ordinate frame of the endoscope's field of view. In another embodiment, a magnetic tracking system is used for the ultrasound probe. Using only one tracking system for both the endoscope and the ultrasound probe reduces obstructions in the environment, and avoids a line-of-sight tracking requirement.

In another embodiment, tracking of the probe is done using an optical tracking system. The calibration of the 3D probe is done in a manner similar to a 2D ultrasound probe calibration using intensity-based registration. Intensity-based registration is fully automatic and does not require segmentation or feature identification. In the typical 2D case, acquired images are subject to scaling in the video generation and capture process. This transformation and the known position of the tracking ultrasonic calibration device (calibration phantom) are used to determine the relationship between the ultrasound imaging volume and the ultrasound probe's tracking device. Successful calibration requires an unchanged geometry. The calibration phantom will be designed to withstand relocation and handling without deformation. A quick-release clamp attached to the phantom will hold the ultrasound probe during the calibration process.

A spatial correlation of the endoscopic video with dynamic ultrasound images is then done. The processing internal to each tracking system, endoscope, and ultrasound machine causes a unique time delay between the real-time input and output of each device. The output data streams are not synchronized and are refreshed at different intervals. In addition, the time taken by the navigation system to acquire and process these outputs is stream-dependant. Consequently, motion due to breathing and other actions can combine with these independent latencies to cause real-time display of dynamic device positions different to those when the imaging is actually being acquired.

In some embodiments, a computer is used to perform the spatial correlation. The computer can handle a larger image volume, allowing for increased size of the physical imaged volume or higher image resolution (up to 512_{—}512_{—}512 instead of 256_{—}256_{—}64). The computer also provides faster 3D reconstruction and merging, and a higher-quality perspective volume rendering at a higher frame rate. The computer time-stamps and buffers the tracking and data streams, and then interpolating tracked device position and orientation to match the image data timestamps.

In determining the required time offset, the ultrasound probe is moved across a step surface in the calibration phantom to create a temporal step function in both the tracking system and image data stream. The relative delay is determined by comparing the timestamps of the observed step function in each data stream. The endoscope latency is determined similarly using the same phantom. In some embodiments, this is done whenever the ultrasound system is reconfigured. The endoscope latency will not need to be recalculated unless the endoscope electronics are changed, however. The patient is imaged through the ultrasound probe, and the endoscope becomes the frame of reference for the surgeon. The important information is contained in the dynamic relationship of the ultrasound data to the endoscope video, which is known through calibration and tracking of both devices.

Turning now to FIG. 1, the process shows (at 120) on a display device one or more images of the patient target site. Next, the process receives (at 125) a user's indication of a spatial feature of the patient target site on the images of the patient target site. The process then projects (at 130) an indicia on the images relating the position and orientation of the surgical instruments to the spatial feature of the patient target site.

The methodology illustrated in FIG. 1 dynamically tracks and targets lesions in motion beyond the visible endoscopic view. When a target is identified, the subregion surrounding the target in the ultrasound volume will be stored as a reference, together with the tracked orientation of the volume. A subregion of each successively-acquired ultrasound volume, centered at the target position in the preceding volume, will be re-sampled using the orientation of the reference target subregion. Three-dimensional cross-correlation of the re-sampled subregion with the reference subregion will be used to find the new location of the target. This dynamic tracking will follow each target over time; if the system is displaying target navigation data, the data will change in real time to follow the updated location of the target relative to the endoscope.

Vascular structures return a strong, well differentiated Doppler signal. The dynamic ultrasound data may be rendered in real time using intensity-based opacity filters, making nonvascular structures transparent. This effectively isolates the vascular structure without requiring computationally-demanding deformable geometric models for segmentation, thus the system can follow movements and deformations in real time.

The methodology illustrated in FIG. 1 allows a user such a surgeon to mark a selected target point or region on intraoperative ultrasonic images (one or more 3-D ultrasound images). The designated target point or region is then displayed to the surgeon during a surgical operation, to guide the position and orientation of the tool toward the target site. In some embodiments, the target area is displayed to the user by (1) displaying a field representing the patient target area, and (2) using the tracked position of the tool with respect to the patient to superimpose on the field one or more indicia whose position in the displayed field is indicative of the relative position of the tool with respect to the marked target position. Also, in some embodiments, the tool is equipped with a laser pointer that directs a laser beam onto the patient to indicate the position and orientation of a trajectory for accessing the target region. The user can follow this trajectory by aligning the tool with the laser-beam.

In the embodiments where the tool is an endoscope, the displayed image is the image seen by the endoscope, and the indicia are displayed on this image. The indicia may indicate target position as the center point of the indicia, e.g., arrows, and tool orientation for reaching the target from that position, by the degree of elongation of arrows, such that the indicia are brought to equal sizes when the tool is properly oriented. Alternatively, the indicia may indicate the surface point for entry and the elongation of the arrows, the tool orientation-trajectory for reaching the target from that surface point.

Some embodiments enable surgeons to visualize a field of view of the surgical endoscope overlaid with volumetrically-reconstructed medical images of a localized area of the patient's anatomy. Using this volumetric navigation system, the surgeon visualizes the surgical site via the surgical endoscope, while exploring the inner layers of the patient's anatomy through the three-dimensionally reconstructed pre-operative MRI or CT images. Given the endoscope's position and orientation, and given the characteristics of the camera, a perspective volume-rendered view matching that of the optical image obtained by the endoscope is rendered. This system allows the surgeon to virtually fly through and around the site of the surgery to visualize alternative approaches and qualitatively determine the best one. The volumetrically reconstructed images are generated using intensity based filtering and direct perspective volume rendering, which removes the need for conventional segmentation of high-contrast images. The real-time 3D-rendered radiographic reconstruction images matched with the intra-operative endoscopic images provide a new capability in minimally-invasive endoscopic surgery. Since hitting vascular structures remains the greatest hazard in endoscopic procedures, this new technology represents a marked improvement over conventional image-guidance systems, which generally display 2D reconstructed images.

In operation, and with respect to embodiments that use ultrasonic images, the user makes a marking on the image corresponding to the target region or site. This marking may be a point, line or area. From this, and by tracking the position of the tool in the patient coordinate system, the system functions to provide the user with visual information indicating the position of the target identified from the ultrasonic image.

The navigation system that uses the process 100 of FIG. 1 operates in three distinct modes. The first is target identification mode. The imaged ultrasound volume will be displayed to allow the surgeon to locate one or more target regions of interest and mark them for targeting. The system will show an interactive volumetric rendering as well as up to three user positionable orthogonal cross-sectional planes for precise 2D location of the target.

In the second mode, the endoscope will be used to set the position and orientation of the frame of reference. Based on these parameters and using the optical characteristics of the endoscope, the system will overlay target navigation data on the endoscope video. This will allow the surgeon to target regions of interest beyond the visual range of the endoscope's field of view. Displayed data will include the directions of, and distances to, the target regions relative to the endoscope tip, as well as a potential range of error in this data.

The third mode will be used to perform the actual interventional procedure (such as biopsy or ablation) once the endoscope is in the correct position. The interactive imaged ultrasound volume and cross-sectional planes will be displayed, with the location of the endoscope and the trajectory through its tip projected onto each of the views. The endoscope needle itself will also be visible in the ultrasound displays.

The navigation system allows the interventional tool to be positioned in the center of the lesion without being limited to a single, fixed 2D ultrasound plane emanating from the endoscope tip. (That 2D view capability can be duplicated by optionally aligning a cross sectional ultrasound plane with the endoscope.) In the first implementation of the endoscope tracking system, a magnetic sensor will need to be removed from the working channel in order to perform the biopsy, and the navigation display will use the stored position observed immediately prior to its removal. In another embodiment, a sensor is integrated into the needle assembly, which will be in place at calibration.

The navigation system provides real-time data on the position and orientation of the endoscope, and the ultrasound system provides the dynamic image data. The tip position data is used to calculate the location of the endoscope tip in the image volume, and the probe orientation data will be used to determine the rendering camera position and orientation. Surgeon feedback will be used to improve and refine the navigation system. Procedure durations and outcomes will be compared to those of the conventional biopsy procedure, performed on the phantom without navigation and image-enhanced endoscopy assistance.

When a target is identified, some embodiments store the subregion surrounding the target in the ultrasound volume as a reference, together with the tracked orientation of the volume. These embodiments will then re-sample a subregion of each successively-acquired ultrasound volume, centered at the target position in the preceding volume, by using the orientation of the reference target subregion.

Some embodiments will use three-dimensional cross-correlation of the re-sampled subregion with the reference subregion to find the new location of the target. This dynamic tracking will follow each target over time; if the system is displaying target navigation data, the data will change in real time to follow the updated location of the target relative to the endoscope.

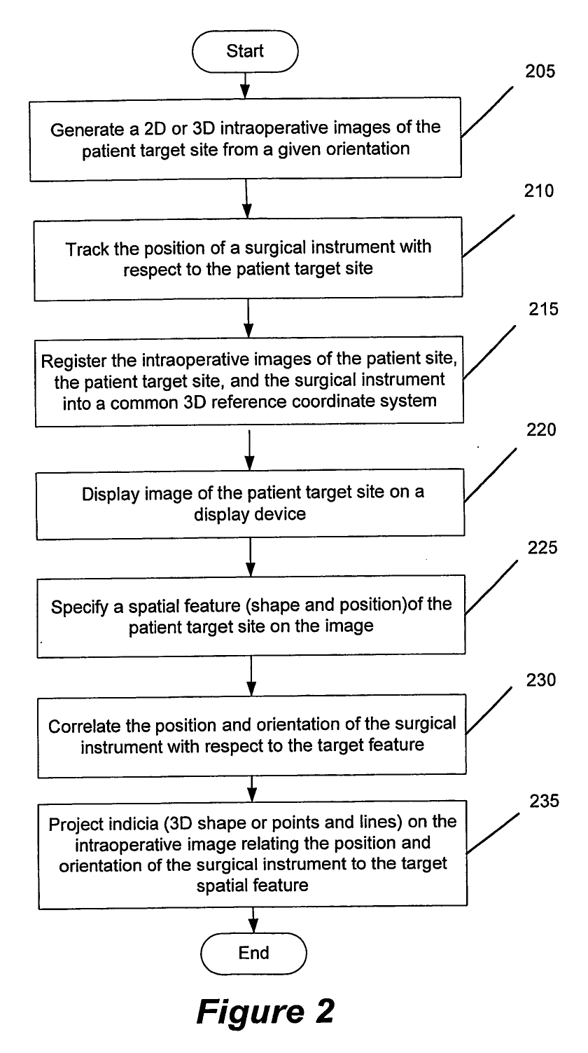

FIG. 2 illustrates a process 200 of some embodiments of the invention. Like the process 100 of FIG. 1, the process 200 guides a medical instrument to a desired position in a patient. As shown FIG. 2, the process 200 initially acquires (at 205) one or more 2D or 3D intraoperative images of the patient target site from a given orientation. Next, the process tracks (at 210) the position of a surgical instrument with respect to the patient target site.

The process then registers (at 215) the intraoperative images of the patient site, the patient target site, and the surgical instrument into a common 3D reference coordinate system. Next, the process renders (at 220) the image of the patient target site on a display device. The process also specifies (at 225) a spatial feature (shape and position) of the patient target site on the image. The process then correlates (at 230) the position and orientation of the surgical instrument with respect to the target feature. The process projects (at 235) an indicia (e.g., a three-dimensional shape, points and/or lines) on the intraoperative image relating the position and orientation of the surgical instrument to the target spatial feature.

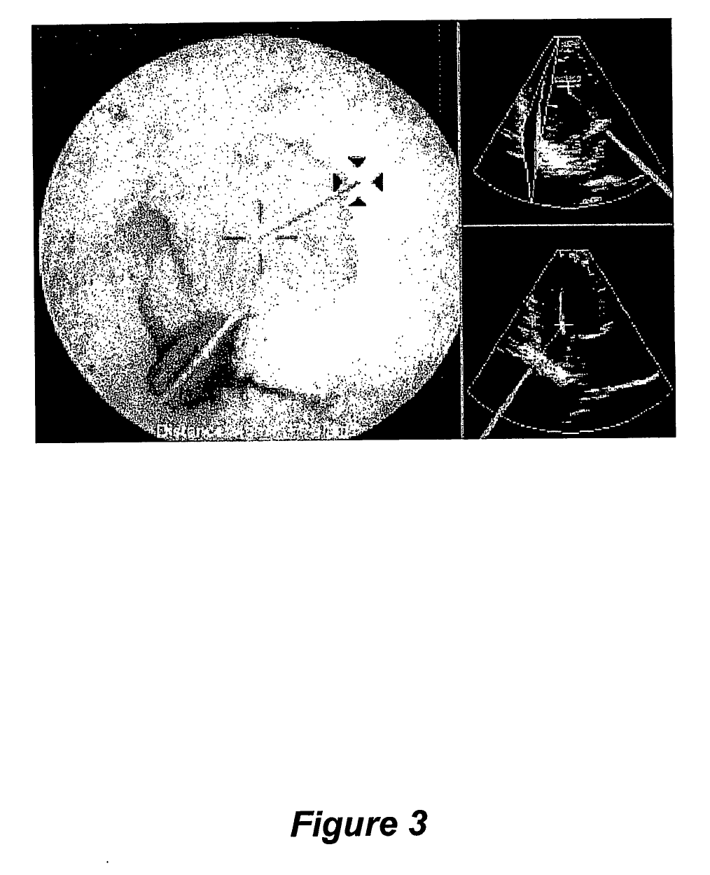

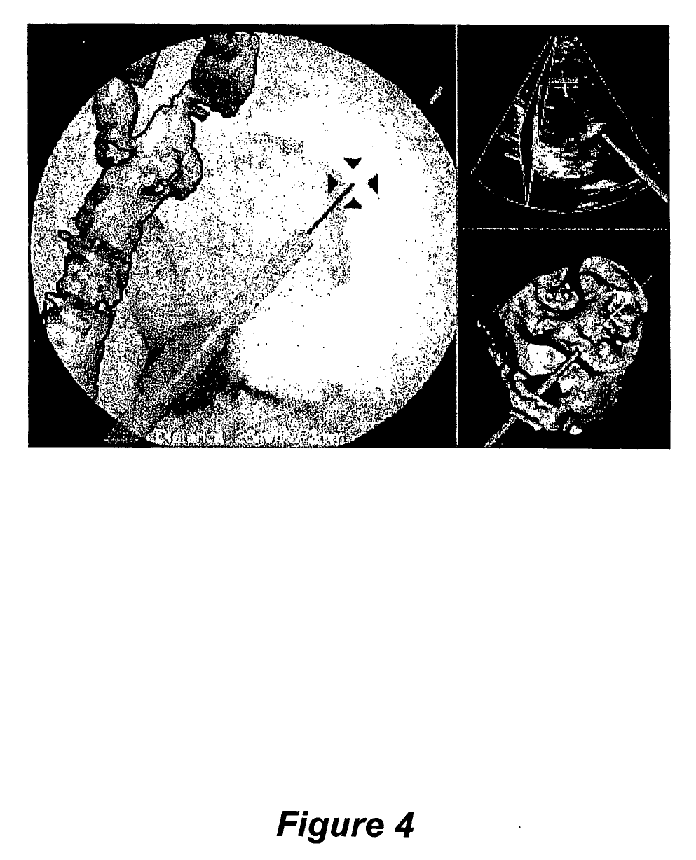

FIGS. 3 and 4 illustrate exemplary user interfaces for the imaging systems that use the processes illustrated in FIGS. 1 and 2. FIG. 3 shows an exemplary user interface (UI) for ultrasound-enhanced endoscopy. The left panel shows the endoscopic view with a superimposed targeting vector and a distance measurement. The right panels show reformatted cross-sectional planes through the acquired 3D ultrasound volume. FIG. 4 shows another UI for ultrasound-enhanced endoscopy. The left panel shows the endoscopic view with virtual tool tracking and visualization and vasculature acquired through Doppler imaging. The lower right panel shows volume-rendered 3D ultrasound.

The UIs of FIGS. 3 and 4 support interactive rendering of the ultrasound data to allow a user to locate and mark the desired region of interest in the ultrasound image volume. The UIs allow the user to locate and mark target regions of interest. Hitting vascular structures is a serious hazard in endoscopic procedures. Visualization of the vasculature behind the surface tissue in the endoscopic view would assist in avoiding the vascular structures (anti-targeting).

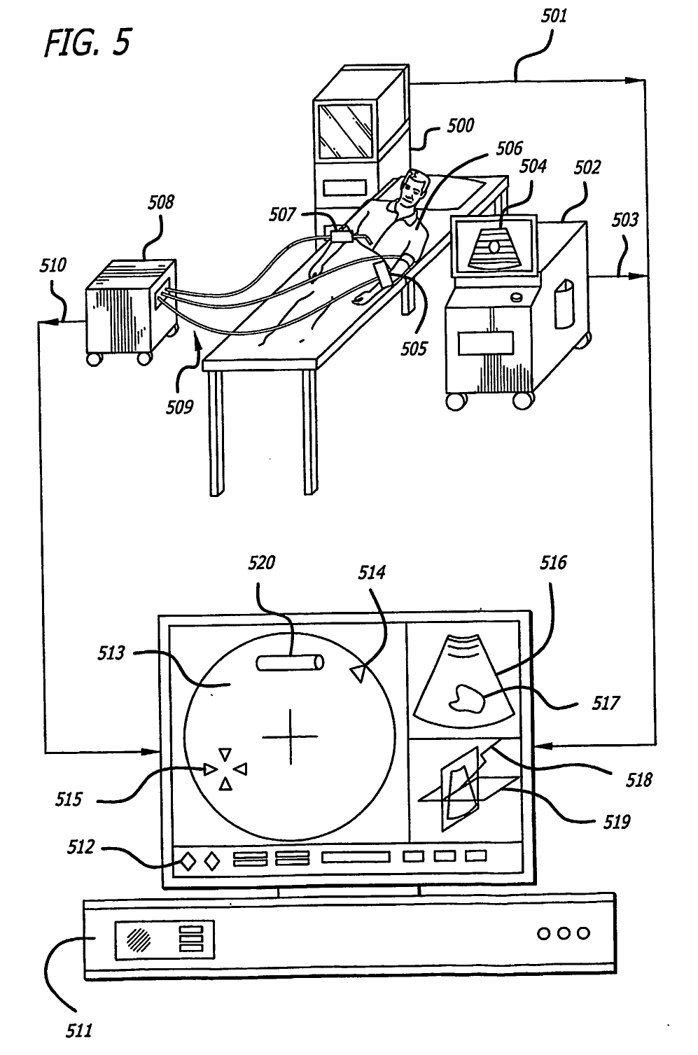

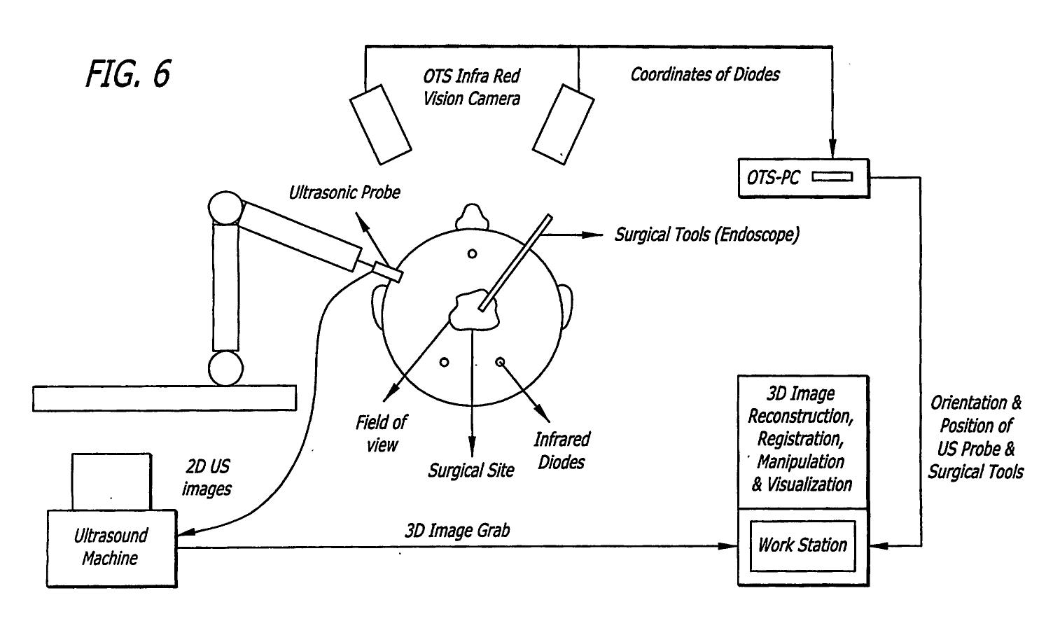

FIGS. 5 and 6 respectively illustrate exemplary surgical arrangements according to some embodiments of the invention. These systems, can:
track 500+ mm flexible endoscopes with an accuracy of 1.8 mm in position and 1° in orientation
acquire external 3D ultrasound images and process them for navigation in near real-time
allow dynamic target identification on any reformatted 3D ultrasound cross-sectional plane view.
optionally overlay dynamic Doppler ultrasound data, rendered using intensity based opacity filters, on the endoscopic view.
provide an overall coarse target accuracy of 10 mm, with a refined target accuracy of 5 mm during breath-holds.

In the system of FIG. 5, a video source 500 (e.g., microscopic or camcorder)is used to generate a video signal 501. In some embodiments described below, the video source 500 is an endoscopic system. An intra-operative imaging system 502 (e.g., an ultrasonic system) captures an intra-operative imaging data stream 103. The information is displayed on an ultrasonic display 504.

A trackable intra-operative imaging probe 505 is also deployed in one or more trackable surgical tools 506. Other tools include a trackable endoscope 507 or any intraoperative video source. The tracking device 508 has tracking wires 509 that communicate a tracking data stream 510. A navigation system 511 with a navigation interface 512 is provided to allow the user to work with an intra-operative video image 513 (perspective view). In the absence of video source this could be blank.

Primary targeting markers 514 (pointing to a target outside the field of view) as well as secondary targeting markers 515 (pointing to a target inside the field of view) can be used. An intra-operative image 516 and an image of the lesion target 517 are shown with a virtual representation of surgical tools or video source 518 (e.g., endoscope) as an orthographic view 519 (outside view). Additionally, an image overlay 520 of any arbitrary 3D shape (anatomical representation or tool representation) can also be shown.

FIG. 6 shows another exemplary surgical set-up. In FIG. 6, several infrared vision cameras capture patient images. An ultrasonic probe positions an ultra-sound sensor in the patient. Surgical tools such as an endoscope are then positioned in the patient. The infrared vision cameras report the position of the sensors to a computer, which in turn forwards the collected information to a workstation that generates a 3D image reconstruction. The workstation also registers, manipulates the data and visualizes the patient data on a screen. The workstation also receives data from an ultrasound machine that captures 2D images of the patient.

Since the geometry of a flexible endoscope in use changes continually, the field of view at the endoscope tip is not directly dependent on the position of a tracking device attached to some other part of the endoscope. This precludes direct optical or mechanical tracking; while useful and accurate, these systems require an uninhibited line of sight or an obtrusive mechanical linkage, and thus cannot be used when tracking a flexible device within the body.

In order to make use of tracked endoscope video, six extrinsic parameters (position and orientation) and five intrinsic parameters (focal length, optical center co-ordinates, aspect ratio, and lens distortion coefficient) of the imaging system are required to determine the pose of the endoscope tip and its optical characteristics. The values of these parameters for any given configuration are initially unknown.

A magnetic transducer is inserted into the working channel at the endoscope tip, positioning the field generator so that the optimal sensing volume encompasses the range of sensor positions. At this time, a 6 DOF miniaturized magnetic tracking system with metal insensitivity is used, although recent developments promise improved systems in the near future.

In order to correctly insert acquired ultrasound images into the volume dataset, the world co-ordinates of each pixel in the image must be determined. This requires precise tracking of the ultrasound probe as well as calibration of the ultrasound image. Current calibration techniques are too cumbersome and time consuming to be performed prior to each use of the 3D ultrasound system.

When tracking ultrasound data, the region of interest may be a significant distance from the probe itself. Consequently, any tracking error is magnified when the probe's orientation is projected to locate the region being imaged.

One of the advantages of the ultrasound reconstruction engine is that it can be adapted to any existing ultrasound system configuration. In order to exploit this versatility, a simple and reliable tracking-sensor mount capability for a variety of types and sizes of ultrasound probes is used, as it is essential that the tracking sensor and ultrasound probe maintain a fixed position relative to each another after calibration. The surgeon may also wish to use the probe independently of the tracking system and its probe attachment.

Accurate volume reconstruction from ultrasound images requires precise estimation of six extrinsic parameters (position and orientation) and any required intrinsic parameters such as scale. The calibration procedure should be not only accurate but also simple and quick, since it should be performed whenever the tracking sensor is mounted on the ultrasound probe or any of the relevant ultrasound imaging parameters, such as imaging depth or frequency of operation, are modified. An optical tracking system is used to measure the position and orientation of a tracking device that will be attached to the ultrasound probe. In order to make the system practical to use in a clinical environment, spatially calibration of the intrinsic and extrinsic parameters of the ultrasound probe is done. These parameters will then be used to properly transform the ultrasound image into the co-ordinate frame of the endoscope's field of view.

The initial solution will be to use magnetic tracking for the ultrasound probe. An alternative solution is to track the probe using an optical tracking system. Tracking devices and a corresponding universal mounting bracket are deployed. In the typical 2D case, acquired images are subject to scaling in the video generation and capture process. Since video output is not used, but the volumetric ultrasound data is accessed directly, this will not be an issue. The intrinsic parameters of the 3D probe, which will have been calibrated by the manufacturer, will be unmodified. A 200_{—}200_{—}200 mm phantom of tissue-mimicking material is used with an integrated CT-visible tracking device. Distributed along all three dimensions within the phantom will be cylinders and cubes, 20 mm in diameter and containing CT contrast material with modified acoustic impedance. The phantom will be imaged using the ultrasound probe; the transformation between the ultrasound volume and a previously acquired, reference CT volumetric image will be computed using intensity-based rigid registration (which requires the intensities of the two images to be similar in structure, but not in value). This transformation and the known position of the phantom's tracking device will be used to determine the relationship between the ultrasound imaging volume and the ultrasound probe's tracking device. Successful calibration requires an unchanged geometry. The phantom will be designed to withstand relocation and handling without deformation. A quick-release clamp attached to the phantom will hold the ultrasound probe during the calibration process.

In order to locate and mark the desired region of interest in the ultrasound image volume, an interface supports interactive rendering of the ultrasound data. An interactive navigation system requires a way for the user to locate and mark target regions of interest. Respiration and other movements will cause the original location of any target to shift. If targets are not dynamically tracked, navigation information will degrade over time. The visibility of regular biopsy needles under ultrasound is poor and hitting vascular structures is a serious hazard in endoscopic procedures. Visualization of the vasculature behind the surface tissue in the endoscopic view would assist in avoiding it (anti-targeting), but segmentation is a difficult, computationally intensive task. To address the foregoing, the navigation system operates in three distinct modes.

The first is target identification mode. The imaged ultrasound volume will be displayed to allow the surgeon to locate one or more target regions of interest and mark them for targeting. The system will show an interactive volumetric rendering as well as up to three user positionable orthogonal cross-sectional planes for precise 2D location of the target.

In the second mode, the endoscope will be used to set the position and orientation of the frame of reference. Based on these parameters and using the optical characteristics of the endoscope, the system will overlay target navigation data on the endoscope video. This will allow the surgeon to target regions of interest beyond the visual range of the endoscope's field of view. Displayed data will include the directions of, and distances to, the target regions relative to the endoscope tip, as well as a potential range of error in this data.

The final mode will be used to perform the actual biopsy once the endoscope is in the correct position. The interactive imaged ultrasound volume and cross-sectional planes will be displayed, with the location of the endoscope and the trajectory through its tip projected onto each of the views. The endoscope needle itself will also be visible in the ultrasound displays.

This will help to position the biopsy needle in the center of the lesion without being limited to a single, fixed 2D ultrasound plane emanating from the endoscope tip, as is currently the case. (That 2D view capability will however be duplicated by optionally aligning a cross-sectional ultrasound plane with the endoscope.) In the first implementation of the flexible endoscope tracking system, the magnetic sensor will need to be removed from the working channel in order to perform the biopsy, and the navigation display will use the stored position observed immediately before its removal. Ultimately, though, a sensor will be integrated into the needle assembly, which will be in place at calibration.

When a target is identified, the subregion surrounding the target in the ultrasound volume will be stored as a reference, together with the tracked orientation of the volume. A subregion of each successively-acquired ultrasound volume, centered at the target position in the preceding volume, will be re-sampled using the orientation of the reference target subregion. Three-dimensional cross-correlation of the re-sampled subregion with the reference subregion will be used to find the new location of the target.

This dynamic tracking will follow each target over time; if the system is displaying target navigation data, the data will change in real time to follow the updated location of the target relative to the endoscope.

Vascular structures return a strong, well differentiated Doppler signal. The dynamic ultrasound data may be rendered in real time using intensity-based opacity filters, making nonvascular structures transparent. This effectively isolates the vascular structure without requiring computationally-demanding deformable geometric models for segmentation, thus being able to follow movement and deformation in real time. If the lag is significant, navigation accuracy will be degraded when the target moves. Where optimal accuracy is required, such as when the actual biopsy is performed, a brief motionless breath-hold may be required.

Lens distortion compensation is performed for the data display in real time, so that the superimposed navigation display maps accurately to the underlying endoscope video.

A new ultrasound volume will replace the next most recent volume in its entirety, much as it does on the display of the ultrasound machine itself, although possibly at a different spatial location. This avoids many problematic areas such as misleading old data, data expiration, unbounded imaging volumes, and locking rendering data. Instead, a simple ping-pong buffer pair may be used; one may be used for navigation and display while the other is being updated. Another benefit of this approach is that the reduced computational complexity contributes to better interactive performance and a smaller memory footprint.

All phantoms will be manufactured to tolerances at least 40 times smaller than the expected system error for the test associated with the phantom. This degree of inaccuracy is small enough to be included in the total system error without any significant impact on specifications.

Computerized object recognition and surveillance are used in order to overlay images of the operative field from ultrasound onto a 3D image of the object using the Laser Targeting System and internal anatomical markers. The 3D images will be created in the workstation both from high resolution MR and CT images and intra-operative ultrasound, obtained with volume acquisition techniques. The system enables an interactive and 3D guidance system for surgeons with maximal flexibility and accuracy. With these techniques, surgery will be performed with the same tools and basic procedures as with non-guided operations, yet the precision and minimization of trauma provided by frame-based stereotaxy.

An exemplary operation of the system will be discussed next help to delineate its features. Take the case of a deep intra-axial brain lesion for which resection is planned. Prior to obtaining the pre-operative images, 4 markers are placed on the patient. These can be small (−2 mm), flashing diodes surrounded by pantopaque filled spheres glued to the skin. Pantopaque is an oil-based, iodine containing X-ray contrast agent that, until recently, was used for myelograms. The iodine makes it visible on CT images while the oil base renders it visible on MRI examinations.

Depending on the imaging characteristics of the lesion and the important surrounding anatomy, (i.e., whether there are calcifications, whether it enhances with gadolinium, etc.) CT or MR scans or both will be performed. Typically high resolution contrast enhanced MR images and MR angiograms would be obtained. The image data would be transferred to the workstation (via the hospital's computer network), volume rendered, and (in the case of multiple imaging modalities) fused.

The image data will be segmented to allow detailed visualization in appropriate anatomic context of the lesion selected intra- and extra-axial structures, and the fiducials. Segmentation of the fiducial markers, brain, the vascular system and surface of the scalp would be fully automatic. The segmentation of the lesion, however, would be only partly automatic, as the irregular anatomy surrounding such lesions is currently too unpredictable for automatic segmentation algorithms.

In the operating room, the patient would be positioned in the usual fashion. The optical tracking system would be positioned above and to the side of the patient's head. Chroma key techniques will automatically identify the flashing markers, enabling automatic and continuous registration and overlay of the patient's physical anatomy with the 3D image data sets. Chroma key is a video special effect technique that allows unique detection of flashing objects with known frequencies in 3D space (e.g., flashing light emitting diodes attached to the patient's head). Diode markers can also be added to conventional ultrasound probes and surgical tools (e.g., probes, scalpels) for their tracking in the stereotactic space. Using chroma key techniques, the markers are automatically recognized and overlaid on the display of the registered 3D image.

A triangle formation of three markers on the ultrasound probe allows the tracking of its movement as it scans the surgical site, thus providing the system with volumetric ultrasound images. Continuous intra-operative registration of the patient's anatomy with the intra-operative ultrasound images is very important due to the movement and deformation of the brain tissue during the surgery. Intra-operatively acquired 3D ultrasound images are then being fused with the pre-operative CT or MR imagery using anatomical features visible to both modalities, such as vascular structures and the lesion. In addition, an extrapolated line extending from the displayed image of a surgical device, indicates the trajectory of the planned approach. Moving the tool automatically leads to a change in the displayed potential trajectory, and the location of the L.E.D. on the tool enables determination of the precise depth and location of the tool, thereby enabling precise determination of the depth and location of the operative site. Therefore, this system not only simplifies the planning of a minimally invasion approach to a direct and interactive task, but it is also more precise than the conventional systems due to its intra-operative image updates and registration using ultrasound imagery.

Furthermore, the information provided by the video camera-based object recognition system, the laser targeting system will further aid in localization of the surgical site, thus increasing the registration and image overlay performance and accuracy by localizing the area for which re-registration is needed. This information allows the workstation to automatically display the real time 3D image of the operative field in context, oriented to (and if desired overlaid with) the approach. The 3D reformatting uses volume display techniques and allows instantaneous variation of transparency. With this technique, deep as well as superficial structures, can be seen in context, thereby considerably enhancing intra-operative guidance.

The system software has two aspects, one dealing with enhancements to the user interface, and the second focusing on algorithms for image manipulation and registration. These algorithms consist of the means for image segmentation, volumetric visualization, image fusion and image overlay. In one embodiment, the system provides:

i) A “user friendly” interface, to facilitate usage in the operating room.

ii) Interactive image analysis and manipulation routines (e.g. arbitrary cuts, image segmentation, image magnification and transformation) on the workstation system.

iii) Seamless Interface between the optical tracking system (provided with a diode encoded pointer) with a computer workstation.

iv) Seamless interface of a video camera and the laser targeting system with the optical tracking system and workstation.

v) Overlay of video images from (iv) on the 3D data from (iii) using diode markers positioned on the surface of the test objects. Test the accuracy of the pointer's guidance in objects.

vi) Inclusion of an ultrasound probe with the optical tracking system and workstation in order to obtain 3D ultrasound images.

vii) Fusion of 3D ultrasound images from (vi) to the 3D MR/CT images from (iii) using diode markers positioned on the ultrasound probe.

viii) Deform test objects after their scan. Attempt to correct the deformation with 3D ultrasound images using linear displacement of the region of interest, then test the accuracy of the pointer's guidance in test objects. Test objects increase in complexity as testing proceeds.

The above-described medical systems have numerous advantages. For instance, these systems enhance intra-operative orientation and exposure in endoscopy, which, in turn, increases surgical precision and speeds convalescence and thereby reduces overall costs. The ultrasound-enhanced endoscopy (USEE) improves localization of targets (e.g., peri-lumenal lesions) that lie hidden beyond endoscopic views. On a single endoscopic view, some of these systems dynamically superimpose directional and targeting information calculated from intra-operative ultrasonic images. Magnetic tracking and 3D ultrasound technologies are used in conjunction with dynamic 3D/video calibration and registration algorithms for precise endoscopic targeting. With USEE, clinicians use the same tools and basic procedures as for current endoscopic operations, but with a higher probability of accurate biopsy, and an increased chance for the complete resection of the abnormality. These systems allow accurate soft-tissue navigation. The systems also provide effective calibration and correlation of intra-operative volumetric imaging data with video endoscopy images.

Some of these systems acquire external 3D ultrasound images and process them for navigation in near real-time. These system allow dynamic target identification on any reformatted 3D ultrasound cross-sectional plane. The system can automatically track the movement of the target as tissue moves or deforms during the procedure. These systems can dynamically map the target location onto the endoscopic view in form of a direction vector and display quantifiable data such as distance to target. Optionally, the systems can provide targeting information on the dynamic 3D ultrasound view. The systems can virtually visualize the position and orientation of tracked surgical tools in the ultrasound view, and optionally also in the endoscopic view. These systems also can overlay dynamic Doppler ultrasound data, rendered using intensity based opacity filters, on the endoscopic view.

The invention has been described in terms of specific examples, which are illustrative only and are not to be construed as limiting. The invention may be implemented in digital electronic circuitry or in computer hardware, firmware, software, or in combinations of them. Apparatus of the invention may be implemented in a computer program product tangibly embodied in a machine-readable storage device for execution by a computer processor; and method steps of the invention may be performed by a computer processor executing a program to perform functions of the invention by operating on input data and generating output. Suitable processors include, by way of example, both general and special purpose microprocessors. Storage devices suitable for tangibly embodying computer program instructions include all forms of non-volatile memory including, but not limited to: semiconductor memory devices such as EPROM, EEPROM, and flash devices; magnetic disks (fixed, floppy, and removable); other magnetic media such as tape; optical media such as CD-ROM disks; and magneto-optic devices. Any of the foregoing may be supplemented by, or incorporated in, specially-designed application-specific integrated circuits (ASICs) or suitably programmed field programmable gate arrays (FPGAs).

From the a foregoing disclosure and certain variations and modifications already disclosed therein for purposes of illustration, it will be evident to one skilled in the relevant art that the present inventive concept can be embodied in forms different from those described and it will be understood that the invention is intended to extend to such further variations. While the preferred forms of the invention have been shown in the drawings and described herein, the invention should not be construed as limited to the specific forms shown and described since variations of the preferred forms will be apparent to those skilled in the art. Thus the scope of the invention is defined by the following claims and their equivalents.

I claim:

1. A method for guiding a medical instrument to a target site within a patient, comprising:
capturing at least one intraoperative ultrasonic image from the patient;
identifying a spatial feature indication of a patient target site on the intraoperative ultrasonic image,
determining coordinates of the patient target site spatial feature in a reference coordinate system,
determining a position of the instrument in the reference coordinate system,
creating a view field from a predetermined position, and optionally orientation, relative to the instrument in the reference coordinate system, and
projecting onto the view field an indicia of the spatial feature of the target site corresponding to the predetermined position, and optionally orientation.

2. The method of claim 1, further comprising
using an ultrasonic source to generate the ultrasonic image of the patient, and
determining coordinates of a spatial feature indicated on said image from the coordinates of the spatial feature on the image and the position, and optionally orientation, of the ultrasonic source.

3. The method of claim 1, wherein said medical instrument is a source of video and the view field projected onto the display device is the image seen by the video source.

4. A computer readable medium that stores a computer program that is designed to assist a user in guiding a medical instrument to a target site in a patient, said computer program comprising sets of instructions for:
capturing at least one image during an operation of the patient;
from a user, receiving an indication of a target site on the captured image;
based on the indication, determining coordinates of the patient target site in a reference coordinate system;
determining a position of the instrument in the reference coordinate system;
projecting onto the display device a field of view from a perspective of the instrument in the reference coordinate system; and
projecting onto the field of view the indicia that specifies the position of the target site relative to the position of the instrument.

5. The computer readable medium of claim 4, wherein the computer program further comprises sets of instructions for:
using an ultrasonic source to generate the ultrasonic image of the patient, and
determining coordinates of a spatial feature indicated on said image from the coordinates of the spatial feature on the image and the position, and optionally orientation, of the ultrasonic source.

6. The computer readable medium of claim 4, wherein said medical instrument is a source of video and the view field projected onto the display device is the image seen by the video source.
